SEQUENCE LISTING (14 sequences)

SEQ ID NO: 1 - DNA, 601 bp, Homo sapiens
ggggaagtgacagtatttttgtatttcatgtaaggaaaacataagccctgaatcgctcacagttattcagtgagagctgggattagaagtcaggaatctcagcttctcatttggcactgtttcttgtaagtacaaaatagttagggaacaaacctccgagatgctacctggataatcaaagattcaaaccaacctcttcaagaagggtgagattccaagataatctcaacctgtctccgcagccccacccatgtgtacccataaaatgaattacacagagatcgctataggatttaaagcktttatactaaatgtgctgggattttgcaaactatagtgtgctgttattgttaatttaaaaaaactctaagttaggattgacaaattatttctctttagtcatttgcttgtatcaccaaagaagcaaacaaacaaacaaacaaaaaaagaaaaagatcttggggatggaaatgttataaagaatcttttttacactagcaatgtctagctgaaggcagatgccctaattccttaatgcagatgctaagagatggcagagttgatcttttatcatctcttggtgaaagcccagtaacataag

SEQ ID NO: 2 - DNA, 801 bp, Homo sapiens
gagatggcagagttgatcttttatcatctcttggtgaaagcccagtaacataagactgctctaggctgtctgcatgcctgtctatctaaattaactagcttggttgctgaacaccaggttaggctctcaaattaccctctgattctgatgtggcctgagtgtgacagttaattattgggaatatcaaaacaattacccagcatgatcatgtattatttaaacagtcctgacagaactgtacctttgtgaacagtgcttttgattgttctacatggcatattcacatccattttcttccacagggtgatcttctgttctagggagaaagtgtctcatttgcagaaaggaaaggcacctgcggtatttgtgaaatgccatgacaagtctctgaataagaagtsaggctggtgagcattctgggctaaagctgactgggcatcctgagcttgcaccctaagggaggcagcttcatgcattcctcttcaccccatcaccagcagcttgccctgactcatgtgatcaaagcattcaatcagtctttcttagtccttctgcatatgtatcaaatgggtctgttgctttatgcaatacttcctctttttttctttctcctcttgtttctcccagcccggaccttcaacccaggcacacattttaggttttattttactccttgaactacccctgaatcttcacttctccttttttctctactgcgtctctgctgactttgcagatgccatctgcagagcatgtaacacaagtttagtagttgccgttctggctgtgggtgcagctctt

SEQ ID NO: 3 - DNA, 801 bp, Homo sapiens
acagtttctttcttctgtgattaaaatcaatcaaatcaaagattctctttctcacaccttctgctaactcctcagaaacctcatatcacaagaaatgaaatggaacaggcctttcgtttgatacattttagaataagaaatcctctaaatttagaagtcatttggcccagtcctccaaaaatgatgcaccttattgggacggggctaaatagttgctccagtgtcttccattcctacaaacctgccattctctgatccattatacacatctcccctgggtttattctcacaacctttgttctgaaattccatttgaaggctttttccatcctaaaaccagtgggggacaggcgggaattgtaaaacactcagaagataataaattgccctttttcctgtgytttttctcagaaactgggcgaatctacagaacaaagaacggcatgtgaattctgtgaagaatgaagtggaggaagtaacttttacaaaacatacccagtgtttggggtgtttcaaaagtggattttcctgaatattaatcccagccctacccttgttagttattttaggagacagtctcaagcactaaaaagtggctaattcaatttatggggtatagtggccaaatagcacatcctccaacgttaaaagacagtggatcatgaaaagtgctgttttgtcctttgagaaagaaataattgtttgagcgcagagtaaaataaggctccttcatgtggcgtattgggccatagcctataattggttagaacctcctattttaattggaattctggatctttc

SEQ ID NO: 4 - DNA, 268 bp, Homo sapiens
ttaatcccagccctacccttgttagttattttaggagacagtctcaagcactaaaaagtggctaattcaatttatggggtatagtggccaaatagcacatcctccaacgttaaaagacagtggatcatgaaaagtgctgttttgtcctttgagaaagaaataattgtttgagcgcagagtaaaataaggctccttcatgtggcgtattgggccatagcctataattggttagaacctcctattttaaytggaattctggatctttcgg

SEQ ID NO: 5 - DNA, 51 bp, Homo sapiens
accgtaattttattattagattctcyaaatgattttcatcaatttaaaatc

SEQ ID NO: 6 - DNA, 601 bp, Homo sapiens
attagattctccaaatgattttcatcaatttaaaatcattcaatatctgacagttactcttcagttttaggcttaccttggtcatgcttcagttgtacttccagtgcgtctcttttgttcctggctttgacatgaaaagataggtttgagttcaaattttgcattgtgtgagcttctacagattttagacaaggaccgtttttactaagtaaaagggtggagaggttcctggggtggattcctaagcagtgcttgtaaaccatcgcgtgcaatgagccagatggagtaccatgagggttgytatttgttgtttttaacaactaatcaagagtgagtgaacaactatttataaactagatctcctatttttcagaatgctcttctacgtataaatatgaaatgataaagatgtcaaatatctcagaggctatagctgggaacccgactgtgaaagtatgtgatatctgaacacatactagaaagctctgcatgtgtgttgtccttcagcataattcggaagggaaaacagtcgatcaagggatgtattggaacatgtcggagtagaaattgttcctgatgtgccagaacttcgaccctttct

SEQ ID NO: 7 - DNA, 203 bp, Homo sapiens
tatttgttgtttttaacaactaatcaagagtgagtgaacaactatttataaactagatctcctatttttcagaatgctcttmtacgtataaatatgaaatgataaagatgtcaaatatctcagaggctatagctgggaacccgactgtgaaagtatgtgatatctgaacacatactagaaagctctgcatgtgtgttgtcctt

SEQ ID NO: 8 - DNA, 601 bp, Homo sapiens
tctcttttgttcctggctttgacatgaaaagataggtttgagttcaaattttgcattgtgtgagcttctacagattttagacaaggaccgtttttactaagtaaaagggtggagaggttcctggggtggattcctaagcagtgcttgtaaaccatcgcgtgcaatgagccagatggagtaccatgagggttgttatttgttgtttttaacaactaatcaagagtgagtgaacaactatttataaactagatctcctatttttcagaatgctcttctacgtataaatatgaaatgataaagrtgtcaaatatctcagaggctatagctgggaacccgactgtgaaagtatgtgatatctgaacacatactagaaagctctgcatgtgtgttgtccttcagcataattcggaagggaaaacagtcgatcaagggatgtattggaacatgtcggagtagaaattgttcctgatgtgccagaacttcgaccctttctctgagagagatgatcgtgcctataaatagtaggaccaatgttgtgattaacatcatcaggcttggaatgaattctctctaaaaataaaatgatgtatgatttgttgtt

SEQ ID NO: 9 - DNA, 401 bp, Homo sapiens
tttgttgttggcatcccctttattaattcattaaatttctggatttgggttgtgacccagggtgcattaacttaaaagattcactaaagcagcacatagcactgggaactctggctccgaaaaactttgttatatatatcaaggatgttctggctttacattttatttattagctgtaaatacatgtgtggatgtgtaaayggagcttgtacatattggaaaggtcattgtggctatctgcatttataaatgtgtggtgctaactgtatgtgtctttatcagtgatggtctcacagagccaactcactcttatgaaatgggctttaacaaaacaagaaagaaacgtacttaactgtgtgaagaaatggaatcagcttttaataaaattgacaacattttat

SEQ ID NO: 10 - DNA, 801 bp, Homo sapiens
  misc_feature (401)..(401): n is a, c, g, or t
aattgttcctgatgtgccagaacttcgaccctttctctgagagagatgatcgtgcctataaatagtaggaccaatgttgtgattaacatcatcaggcttggaatgaattctctctaaaaataaaatgatgtatgatttgttgttggcatcccctttattaattcattaaatttctggatttgggttgtgacccagggtgcattaacttaaaagattcactaaagcagcacatagcactgggaactctggctccgaaaaactttgttatatatatcaaggatgttctggctttacattttatttattagctgtaaatacatgtgtggatgtgtaaatggagcttgtacatattggaaaggtcattgtggctatctgcatttataaatgtgtggtgctaactngtatgtgtctttatcagtgatggtctcacagagccaactcactcttatgaaatgggctttaacaaaacaagaaagaaacgtacttaactgtgtgaagaaatggaatcagcttttaataaaattgacaacattttattaccacactaagtcattattttgtatcatttttaaagtaaatttattcttaggtcagattcactcagcatattttgactaagtaaccactgtacttagtaaaccgaagagcttctgagaattatagtgtaccttatagatatttttaacatttatatttgtataaagctaaagaaagccttacatatcctttaaactgactatagaagaaaatgatacagaattttgcctgcataaagtacacaggactattcttgcctacaat

SEQ ID NO: 11 - DNA, 835 bp, Homo sapiens
actttatgcaggcaaaattctgtatcattttcttctatagtcagtttaaaggatatgtaaggctttctttagctttatacaaatataaatgttaaaaatatctataaggtacactataattctcagaagctctttggtttactaagtacagtggttacttagtcaaaatatgctgagtgaatctgacctaagaataaatttactttaaaaatgatacaaaataatgacttagtgtggtaataaaatgttgtcaattttattaaaagctgattccatttcttcacacagttaagtacgtttctttcttgttttgttaaagcccatttcataagattragttggctctgtgagaccatcactgataaagacacatacagttagcaccacacatttataaatgcagatagccacaatgacctttccaatatgtacaagctccatttacacatccacacatgtatttacagctaataaataaaatgtaaagccagaacatccttgatatatatagcaaagtttttcggagccagagttcccagtgctatgtgctgctttagtgaatcttttaagttaatgcaccctgggtcacaacccaaatccagaaatttaatgaattaataaaggggatgccaacaacaaatcatacatcattttatttttagagagaattcattccaagcctgatgatgttaatcacaacattggtcctactatttataggcacgatcatctctctcagagaaagggtcgaagttctggcacatcaggaacaatttctactccgacatgttccaatacatcccttgatcgactgttttcccttccgaattatgct

SEQ ID NO: 12 - DNA, 833 bp, Homo sapiens
actttatgcaggcaaaattctgtatcattttcttctatagtcagtttaaaggatatgtaaggctttctttagctttatacaaatataaatgttaaaaatatctataaggtacactataattctcagaagctctttggtttactaagtacagtggttacttagtcaaaatatgctgagtgaatctgacctaagaataaatttactttaaaaatgatacaaaataatgacttagtgtggtaataaaatgttgtcaattttattaaaagctgattccatttcttcacacagttaagtacgtttctttcttgttttgttaaagcccatttcataagaktaagttggctctgtgagaccatcactgataaagacacatacagttagcaccacacatttataaatgcagatagccacaatgacctttccaatatgtacaagctccatttacacatccacacatgtatttacagctaataaataaaatgtaaagccagaacatccttgatatatatagcaaagtttttcggagccagagttcccagtgctatgtgctgctttagtgaatcttttaagttaatgcaccctgggtcacaacccaaatccagaaatttaatgaattaataaaggggatgccaacaacaaatcatacatcattttatttttagagagaattcattccaagcctgatgatgttaatcacaacattggtcctactatttataggcacgatcatctctctcagagaaagggtcgaagttctggcacatcaggaacaatttctactccgacatgttccaatacatcccttgatcgactgttttcccttccgaattatg

SEQ ID NO: 13 - DNA, 9734 bp, Homo sapiens
tgtaacacaaaattaaaataagtagaattagttttcagtatttcctatatttggaaaacaatatttatattcattttgtttcttttagttttatttttggcagaactgtaagcaccttcattttctttttcttccaaaggaggagtttaactaccctctggacaatgtccatctcttgggatacagccttggagcccatgctgctggcattgcaggaagtctgaccaataagaaagtcaacagaattactggtaagaaagcaatttcgttggtcttatcataagaggtgaaaagactgtcattctgagagagaatcagaacaaattttgttaaatacccacatgtgtggtgttcttcccggagacatgaccagcacttgattatctcattgtagggctctttattagggataagaaaaaacacagacgctctcactggcttactatccactggcaatagcacagaaataaagcataattacacacaatgcctgcagatttctctgggaagcctgtttcctcccactctcagctctgtgttttagtagtgtaaatgcacatcagtactaggagaaaagaagaaggaccaattccagaggccacttcgaaagaagaccgtcatctaggcaaaggtgtggcatacacacagagagaaagaacccaccactgtttatacatcttctcgacatattcagaaataatctacaaaaggaaatccagccatcctgagtggaaattgctgcataaggctagtttaagagactcaaattcattttagaaggagccaagcctccttttatgtctctctaagtaaagataccatgactgtagaataggagctaataagaatctaaatagctgccagtgcattcaaatgatgagcagtgacatgcgaatgtcatacgaatggaaatttacaaatctgtgttcctgcttttttcccttttaaggcctcgatccagctggacctaactttgagtatgcagaagccccgagtcgtctttctcctgatgatgcagattttgtagacgtcttacacacattcaccagagggtcccctggtcgaagcattggaatccagaaaccagttgggcatgttgacatttacccgaatggaggtacttttcagccaggatgtaacattggagaagctatccgcgtgattgcagagagaggacttggaggtaaatattatttagaagcgaattaaatgtgactcttatccttaacccttattgacccaatgtcctactcagtagcttcaaagtatgtagttttcatatacacatttggccaaattatgtttctgaagaattctgcaatgttcagcatgaccaccttagagccaggcagacagccattttatcttttatttactatactgtaggctacactgagcagtgcacttacagtagcaagagaaaaaggtgggattttagacaggaagactccactgacctcaataatggcatcataaaatgctatctggccacatgttgtcataccttgaatgtagctgcaaagccaatggaaagattttagatgttactggaacagaagatgttaattagcataaatcttccaaaatgttcagaacataatgttagcttaatgttttactttaataatgttagcttgtgttaaatttatgatttttgtttgtttgttttttgagatagagtcttattctattgcccaagctggggtgcagtcacacaatcacagggacttgcaatgttgcccaggctggtctcaaactcctggcctcaagtgatcctcctgcctcagcctcccaaagttctgggattgcagctgtgagccaccacgcccagtttacgatttatttttaagagccccttgcatactttatagacattgggacctacctaggatattctcgttatttttgtgcacgtaatagaacttagagcatattgttactattttcgattgtcctaaaaacttacaaggaattcattcttatggcattgctgattatttctatgttcatttgatataaaagagtgttagtaggggcagaaccctcaattgtacataatatcaatgataaaatacaattcatttaacaattaccctcttaagatgtggtttctagaaatacaaattgtccctaacttacagttttccaactttacaattgggctgtaacaccattttaagttgagaagcacgtgatggtttgacttaaaactttttgacattatgatgggttttgggggtattaagtgcattttgacttacagtatttttgacttatgaagaatttattgtaaggcaaggggcaggtatatgtttctagaagcacctagaagtgttagacactttcaatgtaagagaaggatgagataaacaaggaaatcacacctccaccttggaggcttattacagcttcataaacatactcataaatataagaagcacaaaagtcaaaaattccctgtgaacttgcaactttcactctcttgaaggtgggtgggccgctaccaccaagaatatctcctgaaatagggcctacaatcataaatgcacaggactatatccttgggtgattctactctaacaccacatctcacctattttagacatgccaaatgaaacactctttgtgaatttctgccgagatacaatcttggtgtctcttttttacccagatgtggaccagctagtgaagtgctcccacgagcgctccattcatctcttcatcgactctctgttgaatgaagaaaatccaagtaaggcctacaggtgcagttccaaggaagcctttgagaaagggctctgcttgagttgtagaaagaaccgctgcaacaatctgggctatgagatcaataaagtcagagccaaaagaagcagcaaaatgtacctgaagactcgttctcagatgccctacaaaggtaggctggagactgttgtaaataaggaaaccaaggagtcctatttcatcatgctcactgcatcacatgtactgattctgtccattggaacagagatgatgactggtgttactaaaccctgagccctggtgtttctgttgatagggggttgcattgatccatttgtctgaggcttctaattcccattgtcagcaaggtcccagtgctcagtgtgggatttgcagccttgctcgctgccctcccctgtaaatgtggccattagcatgggctaggctatcagcacagagctcagagctcatttggaaccatccacctcgggtcaacaaactataacccttgtgccaaatccagcctacttcctgcttttgtaaatagtttttttaaaacttttaagttcaggggtacgtatgtaggtttgctaaaaaggtaaacttgtgacatgggagtttgttgtccagaatattccatcacccaggtattaagcttagtacccattagttacttttcctgaagctctccctcctcccaccctctgggaggccccagtgtctgttgttcccctctatgtgctcatgcaaagttttattaggacacagccacacacattcattaccatattgtcaaaggctggtttcatgccaccataacagagttgatagcccacagagcctaaaatatttactccctggccctttacagaatgttcacaacttacataaaggcaaggaccatctgtcttatttatttatttatttaatttgagatgaagtctagctttctcctaggctggaggagaggggcatgatcttggctcaccacaacctctgcctcccgggttcaaatgattcccctgcctcagcctccggagtagctgggataacaggcatgcaccatcatgcccagctaatttttgtatttttagtagagagggggtttcaccgtgttgaccaggctggtctcgaactgctgacctcaggtgatctgccctccttggcctcatctgtctttttaaatgcaactattcctggaaggcaagaatatctcacaccttctaagatactgccattttgccaggagtttgtttcacacttgaatttcaagcttggcctcttgtttagaggcagacctaaaggaatggtcggaaaatgagagaggaggtcttcggataaatccggtgagagggaccaacttcaggaagggtggcttttgtggaatccagatggaaacctgagggaagggatgatattaaagaacagtggccccaggtaaaacatatggcacccatgtgtaaggtgattcttagaatctgtagaggtgtctttcgtggtatagaggttgaggcacctgtgcttcaaggaaaccttaactcttcaaaatcaggcaatgcgtatgaggtaaagagaggactgtgggaccataatcttgaagacacagacaggcttcactcatccctgcctcctgcaccagtgggttcaaggctctgtcagtgtcccctaggggcacctcaccactcccagcttcttcagctctggcctgtcctgctgcctgcaagggttttgcttaattctcaattcaatgtctcttcatcttttagtagctgtggggttttgttgttgttcttctgtttttgcttagtatctgactactttttaattataaaaagagatgtatctaaacaaaatagagattgttatcagaagttcacaacatttattaaaaattttttcacctggacaagagtctaaagcagcataaaaatatggtctgctatattctaaaccatcagtcttaagagatctgtgtctcagcttaagagaaaatacatttaatagacagtaacacaaataagaaaaaaatctgaccaaggatagtgggatatagaagaaaaaacattccaagaattattttatttatttatttatttatttatttatttatttatttatttttgagacacggtctcgctcagttacccaggctggagtgcagcggcgcaatcttaactcactgcaacctctgctttccggttcaagcgattctcctgcctcagcctcctgagtaactgggattacaggcacccgccaccacgcccaactaatttctgtatttttcttagtagaaacagggtttcaccatgttggccaagctagtctcaaactcctgacctcaggtgattcacccaccaaggcctcccaaagtgctgggattacaggcatgagccaccatgcctggcctccaaaaactcttttttcctccatcatcatggttctattttagtcctgctgcctttccttttaacctctccccaggcccatttgctcagggtttttggtagagaccagaggaggggcagggaggagatatagaagttcaactacctgcttccagaggctgtccctagtatagaatactttaggggctggctttacaaggcagtccttgtggcctcactgatggctcaatgaaataagttcttttttaaaaaaaattttatttatttccataggttattgggggaacaggtggtgtttggttacatgagtaagttctttagtagtgatttgtgagattttggtgtgcccattacggaatggaaaaatcaacgaaataagttctatgatgcacctactagacacctaatctgcactagatggtgggggaattaagagcatgggcatgatcctgtgaccggaagcccgcttacagtcagggtggaggacagacctactcatgaaacaaacacagtgacatatagtgacacagaagcaaatgtcaaatatgcttgctccagatgctaaggcacaagatggccaaggatggcggagttcatggagaaagcatcatgagtgttttggccttctgatttgatctccctagcacccctcaaagatggctacttcctaatgctgcttggcaattcagacacatttgggtttttcctatgcatataaccacacttttctgaaagggagtagaattcaaggtctgcattttctaggtatgaacactgtgcatgatgaagtctttccaagccacaccagtggttccatgtgtgtgcacttccggtttgagtgctagtgagatacttctgtggttctgaattgcctgactatttggggttgtgatattttcataaagattgatcaacatgttcgaatttcctccccaacagtcttccattaccaagtaaagattcatttttctgggactgagagtgaaacccataccaatcaggcctttgagatttctctgtatggcaccgtggccgagagtgagaacatcccattcactctgtgagtagcacaggggggcggtcatcatggcaccagtccctcccctgccataacccttggtctgagcagcagaagcagagagcgatgcctagaaaacaagtctttagttaaaaaaatcagaatttcaaaattgaggtctttcctctatttgatattgagaaaaaaatgcttcaaattggccattttattttcacttactagttatatttttttatttatcatcttatatctgtttatttcttttataaagctgctgttaaacaatataattaaactatctcaaaaggtttgacattaaagaaaatgagcaatggtaacaggaaaccactctatagatgtacatataatatgtacagaaaatataagtagtaagaagtccatgacaaagtgttagctctttttttttttttttttttttttttttttgagatggagtctctctcctattgcccaggctggagtgcagtgattcgatctcagctcactgcaacctctacctcccgagttcaaacaattcttctgtctcagcctcccgagtagctggggctgcaggtgcccaccaccatgcccagctaatttttgtatttttagtagcgacagggtctcaccatgttggccaagctggtcttgaattcctgatctcaggtgatccacccgcctcggcctcccaaagtgctgggattacaggtgtgagccaccatgcccagcctaccctttactactaatcaaagaaataaaagtaaggcaacttgatacttttacaattactagatgaacaaatctttaaaaatagccagtgcagacaaggtggtgaagcagaacatgcgaacctaccatgcatcattcacggctagaaccctccaggtgcggaaggtagtattttaataactttccatagctacaaaatattattacatagaagggagtgatttttttctaatatttatcctaaagaaatagtcaacaaacatttttaaaaaacatcaattacagtcgtacctatactagcataaattagaaacccagtatccaacattgaggcagtgggtaaatgaatcgtggtttatcaagtcattaaaatcaatctagcctttaaaaactataattgtaggaaacccaggaaaacatagtaaaaaatggaatataaaatctaaagagaataaagaatagagaatcgtatgtgtgctatgattgtagctaaataatgttcaagtatcaacacaaattgaaaaggaatacatgaaaatgaaaattatatttctgaatgattgacttcaggattttcttttagaattgtattaaatagttcatgtcattaggataaatgctggaatgtggatataatttaaaatatactaaatgccatcgaccttcattttgagttctttgttggacatttttgtgcatttttaaaatatcccctaaataataaagctatttatatttggagaggagaaaaaaaagtggggggcagggagagctgatctctataactaaccaaatttattgcttttttgtttaggcctgaagtttccacaaataagacatactccttcctaatttacacagaggtagatattggagaactactcatgttgaagctcaaatggaagagtgattcatactttagctggtcagactggtggagcagtcccggcttcgccattcagaagatcagagtaaaagcaggagagactcagaaaaagtaattaaatgtatttttcttccttcactttagacccccacctgatgtcaggacctaggggctgtatttcaggggccttcacaattcagggagagctttaggaaaccttgtatttattactgtatgatgtagattttctttaggagtcttcttttattttcttatttttggggggcggggggggaagtgacagtatttttgtatttcatgtaaggaaaacataagccctgaatcgctcacagttattcagtgagagctgggattagaagtcaggaatctcagcttctcatttggcactgtttcttgtaagtacaaaatagttagggaacaaacctccgagatgctacctggataatcaaagattcaaaccaacctcttcaagaagggtgagattccaagataatctcaacctgtctccgcagccccacccatgtgtacccataaaatgaattacacagagatcgctataggatttaaagcttttatactaaatgtgctgggattttgcaaactatagtgtgctgttattgttaatttaaaaaaactctaagttaggattgacaaattatttctctttagtcatttgcttgtatcaccaaagaagcaaacaaacaaacaaaaaaaaaaagaaaaagatcttggggatggaaatgttataaagaatcttttttacactagcaatgtctagctgaaggcagatgccctaattccttaatgcagatgctaagagatggcagagttgatcttttatcatctcttggtgaaagcccagtaacataagactgctctaggctgtctgcatgcctgtctatctaaattaactagcttggttgctgaacaccgggttaggctctcaaattaccctctgattctgatgtggcctgagtgtgacagttaattattgggaatatcaaaacaattacccagcatgatcatgtattatttaaacagtcctgacagaactgtacctttgtgaacagtgcttttgattgttctacatggcatattcacatccattttcttccacagggtgatcttctgttctagggagaaagtgtctcatttgcagaaaggaaaggcacctgcggtatttgtgaaatgccatgacaagtctctgaataagaagtcaggctggtgagcattctgggctaaagctgactgggcatcctgagcttgcaccctaagggaggcagcttcatgcattcctcttcaccccatcaccagcagcttgccctgactcatgtgatcaaagcattcaatcagtctttcttagtccttctgcatatgtatcaaatgggtctgttgctttatgcaatacttcctctttttttctttctcctcttgtttctcccagcccggaccttcaacccaggcacacattttaggttttattttactccttgaactacccctgaatcttcacttctccttttttctctactgcgtctctgctgactttgcagatgccatctgcagagcatgtaacacaagtttagtagttgccgttctggctgtgggtgcagctcttcccaggatgtattcagggaagtaaaaagatctcactgcatcacctgcagccacatagttcttgattctccaagtgccagcatactccgggacacacagccaacagggctgccccaagcacccatctcaaaaccctcaaagctgccaagcaaacagaatgagagttataggaaactgttctctcttctatctccaaacaactctgtgcctctttcctacctgacctttagggctaatccatgtggcagctgttagctgcatctttccagagcgtcagtactgagaggacactaag

SEQ ID NO: 14 - protein, 475 aa, Homo sapiens
MESKALLVLTLAVWLQSLTASRGGVAAADQRRDFIDIESKFALRTPEDTAEDTCHLIPGVAESVATCHFNHSSKTFMVIHGWTVTGMYESWVPKLVAALYKREPDSNVIVVDWLSRAQEHYPVSAGYTKLVGQDVARFINWMEEEFNYPLDNVHLLGYSLGAHAAGIAGSLTNKKVNRITGLDPAGPNFEYAEAPSRLSPDDADFVDVLHTFTRGSPGRSIGIQKPVGHVDIYPNGGTFQPGCNIGEAIRVIAERGLGDVDQLVKCSHERSIHLFIDSLLNEENPSKAYRCSSKEAFEKGLCLSCRKNRCNNLGYEINKVRAKRSSKMYLKTRSQMPYKVFHYQVKIHFSGTESETHTNQAFEISLYGTVAESENIPFTLPEVSTNKTYSFLIYTEVDIGELLMLKLKWKSDSYFSWSDWWSSPGFAIQKIRVKAGETQKKVIFCSREKVSHLQKGKAPAVFVKCHDKSLNKKSG

(19) United States
(12) Patent Application Publication
(10) Pub. No.: US 2010/0130600 A1
(43) Pub. Date: May 27, 2010

(54) LIPOPROTEIN LIPASE AND ITS EFFECT ON STATIN TREATMENTS

(75) Inventors: Mark O. Goodarzi, Los Angeles, CA (US); Kent D. Taylor, Ventura, CA (US); Maren T. Scheuner, Manhattan Beach, CA (US); Xiuqing Guo, Santa Monica, CA (US); Prediman K. Shah, Los Angeles, CA (US); Jerome I. Rotter, Los Angeles, CA (US)

Correspondence Address: DAVIS WRIGHT TREMAINE LLP/Los Angeles, 865 FIGUEROA STREET, SUITE 2400, LOS ANGELES, CA, 90017-2566, US

(73) Assignee: CEDARS-SINAI MEDICAL CENTER, Los Angeles, CA (US)

(21) Appl. No.: 12/594,143

(22) PCT Filed: Mar. 28, 2008
(86) PCT No.: PCT/US08/58623

§ 371 Date: Oct. 7, 2009

Related U.S. Application Data
(60) Provisional application No. 60/909,294, filed on Mar. 30, 2007.

(51) Int. Cl. C12Q 1/68; A61K 31/366

(52) U.S. Cl. 514/460; 435/6

(57) ABSTRACT

The invention provides methods of treating and prognosing atherosclerosis and lipid response to statin treatment by determining the presence or absence of haplotypes at the lipoprotein lipase locus. In one embodiment, the invention is practiced by evaluating the prognosis of vascular grafts in an individual undergoing statin treatment by determining the presence or absence of haplotypes at the lipoprotein locus.

### GOVERNMENT RIGHTS

This invention was made with U.S. Government support, supported by National Institutes of Health Grant HL-69757 and Pharmacogenetic Network Grant HL-69757. The U.S. Government may have certain rights in this invention.

### FIELD OF THE INVENTION

The invention relates generally to the fields of metabolism and, more specifically, to genetic methods of determining lipid metabolism.

### BACKGROUND

All publications herein are incorporated by reference to the same extent as if each individual publication or patent application was specifically and individually indicated to be incorporated by reference. The following description includes information that may be useful in understanding the present invention. It is not an admission that any of the information provided herein is prior art or relevant to the presently claimed invention, or that any publication specifically or implicitly referenced is prior art.

Atorvastatin, pravastatin, simvastatin, cerivastatin, fluvastatin, and lovastatin are all examples of a class of drugs called statins, known for lowering the amount of lipid in the blood and effectively reducing the primary and secondary risk of coronary artery disease. The administering of statins has also been found to decrease the need for coronary artery bypass graft, as well as help the long term outcome of coronary artery bypass graft subjects. Coronary artery bypass graft is a surgical intervention for those who develop atherosclerotic occlusion in coronary arteries, a procedure where the subject's own saphenous vein or brachial or mammary artery is used to bypass the problematic coronary artery. Although statin and coronary artery bypass graft are both commonly used treatments and procedures with often beneficial results for the patient, there remains a substantial percentage of patients who have complications associated with the procedure, or are generally unresponsive to treatment.

As a key regulator of lipid metabolism, genetic variants in the lipoprotein lipase gene (“LPL”) may influence the response to lipid lowering drug therapy. However, very few studies in the past have examined this question. The Regression Growth Evaluation Statin Study (“REGRESS”) found that the D9N variant in LPL attenuated the total cholesterol and low density lipoprotein cholesterol (“LDL-C”) response to pravastatin, but had no significant effect on angiographic progression of coronary artery lesions (Jukema J W, Circulation 1996, 94: 1913-1918). In initial studies in the Post-Coronary Artery Bypass Graft Trial (Post-CABG Trial) cohort, no effect of D9N was observed whereas the HindIII variant in LPL was associated with increased coronary graft narrowing over time, independent of the degree of lipid lowering (moderate versus aggressive) with lovastatin (Taylor K D, Genet Med 2004, 6: 481-486; The Post Coronary Artery Bypass Graft Trial Investigators, N Engl J Med 1997, 336:153-162). Although there have been some associations found between risk factors and atherosclerosis and lipid response to statin therapy, the exact cause and contribution factors are largely unknown. Thus, there is need in the art to determine genes, allelic variants, biological pathways, and other factors that influence lipoprotein cholesterol response to lipid lowering therapy, including but not limited to statin therapy and progression of atherosclerosis in coronary artery bypass grafts.

### SUMMARY OF THE INVENTION

Various embodiments provide methods for evaluating the prognosis of vascular grafts in an individual undergoing statin treatment, comprising obtaining a DNA sample from the individual, and analyzing the DNA sample for at least one haplotype of a human gene coding lipoprotein lipase (“LPL”), the at least one haplotype selected from the group consisting of haplotype 1, haplotype 6, haplotype 7, haplotype 8, haplotype 2 and haplotype 4, where the presence of haplotype 1, haplotype 6, haplotype 7, and/or haplotype 8 is indicative of a favorable prognosis, and where the presence of haplotype 2 and/or haplotype 4 is indicative of an unfavorable prognosis. In other embodiments, the at least one haplotype comprises SEQ. ID. NO.: 1, SEQ. ID. NO.: 2, SEQ. ID. NO.: 3, SEQ. ID. NO.: 4, SEQ. ID. NO.: 5, SEQ. ID. NO.: 6, SEQ. ID. NO.: 7, SEQ. ID. NO.: 8, SEQ. ID. NO.: 9, SEQ. ID. NO.: 10, SEQ. ID. NO.: 11, and/or SEQ. ID. NO.: 12.

Other embodiments provide methods of determining the prognosis of atherosclerosis in coronary grafts in an individual undergoing statin treatment, comprising determining the presence or absence one or more haplotype at the lipoprotein lipase (“LPL”) locus selected from the group consisting of haplotype 1, haplotype 6, haplotype 7, and haplotype 8, determining an increase or decrease in lipid level by comparing a baseline measurement with a follow-up measurement, and prognosing an uncomplicated case of atherosclerosis in coronary grafts if the individual undergoing statin treatment demonstrates the presence of one of the one or more haplotype at the LPL locus and/or an increase in lipid level. In another embodiment, the lipid level comprises HDL-cholesterol. In another embodiment, the statin is lovastatin.

Other embodiments provide methods of determining the prognosis of atherosclerosis in an individual undergoing statin treatment, comprising determining the presence or absence of one or more haplotypes at the lipoprotein lipase (“LPL”) locus selected from the group consisting of haplotype 2 and haplotype 4, determining an increase or decrease in lipid response to statin treatment by comparing a baseline measurement with a follow-up measurement, and prognosing a complicated case of atherosclerosis if the individual undergoing statin treatment demonstrates the presence of one of the one or more haplotypes at the LPL locus and/or a decrease in lipid response to statin treatment. In other embodiments, the lipid comprises triglyceride. In other embodiments, the lipid comprises HDL-cholesterol. In other embodiments, the one or more haplotypes at the LPL locus comprise one or more variant alleles selected from SEQ. ID. NO.: 1, SEQ. ID. NO.: 2, SEQ. ID. NO.: 3, SEQ. ID. NO.: 4, SEQ. ID. NO.: 5, SEQ. ID. NO.: 6, SEQ. ID. NO.: 7, SEQ. ID. NO.: 8, SEQ. ID. NO.: 9, SEQ. ID. NO.: 10, SEQ. ID. NO.: 11, and SEQ. ID. NO.: 12.

Various embodiments also provide methods of treating atherosclerosis in an individual, comprising determining the presence of at least one haplotype at the lipoprotein lipase locus selected from the group consisting of haplotype 2 and haplotype 4, and treating the atherosclerosis in the individual.

Other embodiments provide methods of diagnosing susceptibility to vascular graft occlusion in an individual, comprising determining the presence or absence of haplotype 2 at the lipoprotein lipase locus and/or haplotype 4 at the lipoprotein lipase locus, and diagnosing susceptibility to vascular graft occlusion based upon the presence of haplotype 2 at the lipoprotein lipase locus and/or haplotype 4 at the lipoprotein lipase locus.

Other embodiments provide methods of diagnosing a low probability of vascular graft occlusion in an individual, comprising determining the presence or absence of haplotype 1 at the lipoprotein lipase locus, and diagnosing a low probability of vascular graft occlusion based upon the presence of haplotype 1. In other embodiments, haplotype 1 comprises one or more variant alleles selected from SEQ. ID. NO.: 1, SEQ. ID. NO.: 2, SEQ. ID. NO.: 3, SEQ. ID. NO.: 4, SEQ. ID. NO.: 5, SEQ. ID. NO.: 6, SEQ. ID. NO.: 7, SEQ. ID. NO.: 8, SEQ. ID. NO.: 9, SEQ. ID. NO.: 10, SEQ. ID. NO.: 11, and SEQ. ID. NO.: 12.

Other features and advantages of the invention will become apparent from the following detailed description, taken in conjunction with the accompanying drawing, which illustrate, by way of example, various embodiments of the invention.

### DESCRIPTION OF THE INVENTION

All references cited herein are incorporated by reference in their entirety as though fully set forth. Unless defined otherwise, technical and scientific terms used herein have the same meaning as commonly understood by one of ordinary skill in the art to which this invention belongs. Singleton et al., Dictionary of Microbiology and Molecular Biology 3^{rd} ed., J. Wiley & Sons (New York, N.Y. 2001); March, Advanced Organic Chemistry Reactions, Mechanisms and Structure 5^{th} ed., J. Wiley & Sons (New York, N.Y. 2001); and Sambrook and Russel, Molecular Cloning: A Laboratory Manual 3rd ed., Cold Spring Harbor Laboratory Press (Cold Spring Harbor, N.Y. 2001), provide one skilled in the art with a general guide to many of the terms used in the present application.

One skilled in the art will recognize many methods and materials similar or equivalent to those described herein, which could be used in the practice of the present invention. Indeed, the present invention is in no way limited to the methods and materials described.

“SNP” as used herein means single nucleotide polymorphism.

“DBP” as used herein means diastolic blood pressure.

“HDL-C” as used herein means high density lipoprotein cholesterol.

“HMG-CoA” as used herein means 3-hydroxy-3-methylglutaryl-Coenzyme A.

“LDLC” as used herein means low density lipoprotein cholesterol.

“LPL” as used herein means lipoprotein lipase.

“CABG” as used herein means coronary artery bypass graft.

“SBP” as used herein means systolic blood pressure.

“Haplotype” as used herein refers to a set of single nucleotide polymorphisms (SNPs) on a gene or chromatid that are statistically associated.

“Baseline measurement” as used herein refers to an initial measurement in an individual, taken during the course of a study, so that from future measurements in the individual, one would be able to determine whether there is an increase or decrease in measurement.

“Follow-up measurement” as used herein refers to a measurement taken after a baseline measurement, so that by comparing the follow-up measurement with the baseline measurement, one could determine whether there is an increase or decrease in measurement.

The identities of the LPL haplotypes and markers, their location on the gene and their nucleotide substitutions may be found in Tables 2 and 3. As described herein, 12 single nucleotide polymorphisms (“SNPs”) were genotyped in the lipoprotein lipase gene. From the 12 SNPs, haplotypes were constructed as described in Table 3. As used herein, haplotypes 12-1, 12-2, 12-3, 12-4, 12-5, 12-6, 12-7, 12-8, 12-9, 12-10, 12-11 and 12-12, are also called haplotypes 1, 2, 3, 4, 5, 6, 7, 8, 9, 10, 11 and 12, respectively.

Examples of rs320, rs328, rs11570891, rs3289, rs1803924, rs1059507, rs3735964, rs3200218, rs1059611, rs10645926, rs15285, rs3866471 are described herein as SEQ. ID. NOS: 1-12, respectively.

An example of an LPL gene is described herein as SEQ. ID. NO.: 13, and an example of LPL expressed as a peptide is described herein as SEQ. ID. NO.: 14.

As used herein, the term “MACAD” means the Mexican-American Coronary Artery Disease project, a study aimed at identifying genes common to insulin resistance and atherosclerosis.

As used herein, the term “biological sample” means any biological material from which nucleic acid molecules can be prepared. As non-limiting examples, the term material encompasses whole blood, plasma, saliva, cheek swab, or other bodily fluid or tissue that contains nucleic acid.

The methods may include the steps of obtaining a biological sample containing nucleic acid from the individual and determining the presence or absence of a SNP and/or a haplotype in the biological sample.

As described herein, the inventors asked whether LPL haplotypes influence response to lipid-lowering therapy. They studied 830 subjects from the Post-Coronary Artery Bypass Graft trial, in which subjects with at least one patent saphenous vein graft were treated moderately or aggressively with lovastatin. A lipid profile was obtained at baseline and 4-5 years after treatment. 12 SNPs spanning the 3′ end of LPL were genotyped using the 5′-exonuclease (Taqman MBG) reaction. Haplotypes were constructed using the accelerated expectation maximization algorithm implemented in the program Haploview. Association with lipid response was evaluated using analysis of covariance (ANCOVA). Age, body mass index, race, current smoking status, time between CABG and study enrollment, and lovastatin treatment group were taken as covariates in the association analyses. The inventors observed the same LPL 3′ end haplotypes in a mostly Caucasian population as seen in other populations. The fourth most frequent haplotype (Haplotype 4) was associated with a decreased increment in HDL-cholesterol (Haplotype 4 carriers: +6.8% HDL-C response vs. non-carriers: +14.4% HDL-C response, P=0.005). Conversely, Haplotypes 6, 7, and 8 were each associated with increased HDL-C response to therapy compared to respective non-carriers. Haplotype 2 was associated with a smaller increment in triglycerides (Haplotype 2 carriers: +2.6% versus non-carriers: +11.8% change in triglycerides, P=0.02). These effects of LPL haplotypes on HDL-C and triglyceride response were independent of whether subjects were in the intensive or moderate treatment group. Haplotype 4 exhibited a deleterious effect on HDL-C response to lovastatin therapy, consistent with prior observations of haplotype 4 as predisposing to coronary artery disease, insulin resistance, increased body mass index and increased blood pressure. The most common haplotype, haplotype 1, was protective against graft worsening or occlusion. LPL may influence atherosclerosis risk through pleiotropic effects on each aspect of the metabolic syndrome.

In one embodiment, the present invention provides a method determining a favorable prognosis for vascular graphs in patients undergoing statin treatment by determining the presence or absence of variants at the lipoprotein lipase locus, where the presence of haplotype 1, haplotype 6, haplotype 7, and/or haplotype 8 at the lipoprotein lipase locus is indicative of a favorable prognosis. In another embodiment, the presence of haplotype 1 is indicative of a decrease in vascular graft occlusion. In another embodiment, the presence of haplotype 6, haplotype 7 and/or haplotype 8 is indicative of an increase in HDL-cholesterol response to statin treatment.

In another embodiment, the present invention provides a method of treatment of atherosclerosis by determining the presence of haplotype 1, haplotype 6, haplotype 7, and/or haplotype 8 at the lipoprotein lipase locus and treating the atherosclerosis.

In one embodiment, the present invention provides a method determining an unfavorable prognosis for vascular graphs in patients undergoing statin treatment by determining the presence or absence of variants at the lipoprotein lipase locus, where the presence of haplotype 2 and/or haplotype 4 at the lipoprotein lipase locus is indicative of an unfavorable prognosis. In another embodiment, the presence of haplotype 2 is indicative of lowering of triglyceride response to statin treatment. In another embodiment, the presence of haplotype 4 is indicative of decreasing HDL-cholesterol response to statin treatment.

In another embodiment, the present invention provides a method of treatment of atherosclerosis by determining the presence of haplotype 2 and/or haplotype 4 at the lipoprotein lipase locus and treating the atherosclerosis.

Variety of Methods and Materials

A variety of methods can be used to determine the presence or absence of a variant allele or haplotype. As an example, enzymatic amplification of nucleic acid from an individual may be used to obtain nucleic acid for subsequent analysis. The presence or absence of a variant allele or haplotype may also be determined directly from the individual's nucleic acid without enzymatic amplification.

Analysis of the nucleic acid from an individual, whether amplified or not, may be performed using any of various techniques. Useful techniques include, without limitation, polymerase chain reaction based analysis, sequence analysis and electrophoretic analysis. As used herein, the term “nucleic acid” means a polynucleotide such as a single or double-stranded DNA or RNA molecule including, for example, genomic DNA, cDNA and mRNA. The term nucleic acid encompasses nucleic acid molecules of both natural and synthetic origin as well as molecules of linear, circular or branched configuration representing either the sense or antisense strand, or both, of a native nucleic acid molecule.

The presence or absence of a variant allele or haplotype may involve amplification of an individual's nucleic acid by the polymerase chain reaction. Use of the polymerase chain reaction for the amplification of nucleic acids is well known in the art (see, for example, Mullis et al. (Eds.), The Polymerase Chain Reaction, Birkhauser, Boston, (1994)).

A TaqmanB allelic discrimination assay available from Applied Biosystems may be useful for determining the presence or absence of a variant allele. In a TaqmanB allelic discrimination assay, a specific, fluorescent, dye-labeled probe for each allele is constructed. The probes contain different fluorescent reporter dyes such as FAM and VICTM to differentiate the amplification of each allele. In addition, each probe has a quencher dye at one end which quenches fluorescence by fluorescence resonant energy transfer (FRET). During PCR, each probe anneals specifically to complementary sequences in the nucleic acid from the individual. The 5′ nuclease activity of Taq polymerase is used to cleave only probe that hybridize to the allele. Cleavage separates the reporter dye from the quencher dye, resulting in increased fluorescence by the reporter dye. Thus, the fluorescence signal generated by PCR amplification indicates which alleles are present in the sample. Mismatches between a probe and allele reduce the efficiency of both probe hybridization and cleavage by Taq polymerase, resulting in little to no fluorescent signal. Improved specificity in allelic discrimination assays can be achieved by conjugating a DNA minor grove binder (MGB) group to a DNA probe as described, for example, in Kutyavin et al., “3′-minor groove binder-DNA probes increase sequence specificity at PCR extension temperature, “Nucleic Acids Research 28:655-661 (2000)). Minor grove binders include, but are not limited to, compounds such as dihydrocyclopyrroloindole tripeptide (DPI,).

Restriction fragment length polymorphism (RFLP) analysis may also be useful for determining the presence or absence of a particular allele (Jarcho et al. in Dracopoli et al., Current Protocols in Human Genetics pages 2.7.1-2.7.5, John Wiley & Sons, New York; Innis et al.,(Ed.), PCR Protocols, San Diego: Academic Press, Inc. (1990)). As used herein, restriction fragment length polymorphism analysis is any method for distinguishing genetic polymorphisms using a restriction enzyme, which is an endonuclease that catalyzes the degradation of nucleic acid and recognizes a specific base sequence, generally a palindrome or inverted repeat. One skilled in the art understands that the use of RFLP analysis depends upon an enzyme that can differentiate two alleles at a polymorphic site.

Allele-specific oligonucleotide hybridization may also be used to detect a disease-predisposing allele. Allele-specific oligonucleotide hybridization is based on the use of a labeled oligonucleotide probe having a sequence perfectly complementary, for example, to the sequence encompassing a disease-predisposing allele. Under appropriate conditions, the allele-specific probe hybridizes to a nucleic acid containing the disease-predisposing allele but does not hybridize to the one or more other alleles, which have one or more nucleotide mismatches as compared to the probe. If desired, a second allele-specific oligonucleotide probe that matches an alternate allele also can be used. Similarly, the technique of allele-specific oligonucleotide amplification can be used to selectively amplify, for example, a disease-predisposing allele by using an allele-specific oligonucleotide primer that is perfectly complementary to the nucleotide sequence of the disease-predisposing allele but which has one or more mismatches as compared to other alleles (Mullis et al., supra, (1994)). One skilled in the art understands that the one or more nucleotide mismatches that distinguish between the disease-predisposing allele and one or more other alleles are preferably located in the center of an allele-specific oligonucleotide primer to be used in allele-specific oligonucleotide hybridization. In contrast, an allele-specific oligonucleotide primer to be used in PCR amplification preferably contains the one or more nucleotide mismatches that distinguish between the disease-associated and other alleles at the 3′ end of the primer.

A heteroduplex mobility assay (HMA) is another well known assay that may be used to detect a SNP or a haplotype. HMA is useful for detecting the presence of a polymorphic sequence since a DNA duplex carrying a mismatch has reduced mobility in a polyacrylamide gel compared to the mobility of a perfectly base-paired duplex (Delwart et al., Science 262:1257-1261 (1993); White et al., Genomics 12:301-306 (1992)).

The technique of single strand conformational, polymorphism (SSCP) also may be used to detect the presence or absence of a SNP and/or a haplotype (see Hayashi, K., Methods Applic. 1:34-38 (1991)). This technique can be used to detect mutations based on differences in the secondary structure of single-strand DNA that produce an altered electrophoretic mobility upon non-denaturing gel electrophoresis. Polymorphic fragments are detected by comparison of the electrophoretic pattern of the test fragment to corresponding standard fragments containing known alleles.

Denaturing gradient gel electrophoresis (DGGE) also may be used to detect a SNP and/or a haplotype. In DGGE, double-stranded DNA is electrophoresed in a gel containing an increasing concentration of denaturant; double-stranded fragments made up of mismatched alleles have segments that melt more rapidly, causing such fragments to migrate differently as compared to perfectly complementary sequences (Sheffield et al., “Identifying DNA Polymorphisms by Denaturing Gradient Gel Electrophoresis” in Innis et al., supra, 1990).

Other molecular methods useful for determining the presence or absence of a SNP and/or a haplotype are known in the art and useful in the methods of the invention. Other well-known approaches for determining the presence or absence of a SNP and/or a haplotype include automated sequencing and RNAase mismatch techniques (Winter et al., Proc. Natl. Acad. Sci. 82:7575-7579 (1985)). Furthermore, one skilled in the art understands that, where the presence or absence of multiple alleles or haplotype(s) is to be determined, individual alleles can be detected by any combination of molecular methods. See, in general, Birren et al. (Eds.) Genome Analysis: A Laboratory Manual Volume 1 (Analyzing DNA) New York, Cold Spring Harbor Laboratory Press (1997). In addition, one skilled in the art understands that multiple alleles can be detected in individual reactions or in a single reaction (a “multiplex” assay). In view of the above, one skilled in the art realizes that the methods of the present invention for diagnosing or predicting susceptibility to or protection against various genetic disorders in an individual may be practiced using one or any combination of the well known assays described above or another art-recognized genetic assay.

One skilled in the art will recognize many methods and materials similar or equivalent to those described herein, which could be used in the practice of the present invention. Indeed, the present invention is in no way limited to the methods and materials described. For purposes of the present invention, the following terms are defined below.

### EXAMPLES

The following examples are provided to better illustrate the claimed invention and are not to be interpreted as limiting the scope of the invention. To the extent that specific materials are mentioned, it is merely for purposes of illustration and is not intended to limit the invention. One skilled in the art may develop equivalent means or reactants without the exercise of inventive capacity and without departing from the scope of the invention.

### Example 1

### Results

The clinical characteristics of 891 fully phenotyped subjects are shown in Table 1. In the Post-CABG trial, an overall 15% reduction in total cholesterol (TC) and a 26% reduction in LDL-C was observed. The aggressive treatment group had significantly greater reductions in these parameters than the moderate treatment group (TC: 23% versus 7%; LDL-C: 37% versus 14%, both P<0.0001). Response in high density lipoprotein cholesterol (HDL-C) and triglycerides (TG) did not differ between the two treatment groups. A wide range of lipid responses was observed. Gender or race did not influence lipid response.

The inventors genotyped 12 single nucleotide polymorphisms (SNPs) in the LPL gene. Table 2 shows the frequency and position information of the 12 LPL variants based on genotyping in all 903 subjects. The inventors were able to successfully genotype and assign a common haplotype to 829 of the phenotyped and genotyped subjects. Linkage disequilibrium (D′) among the 12 SNPs (the HindIII variant plus 11 additional SNPs) ranged from 0.55 to 1 (average D′ of 0.92). The haplotypes constructed based on these 12 variants are listed in Table 3, along with their respective frequencies. These haplotypes are labeled 12-1, 12-2, 12-3, etc. to denote that they are based on a total of 12 polymorphisms, and to avoid confusion with the previously reported 19-SNP based haplotypes. The eight most frequent haplotypes together comprise 96% of the haplotypes found in this population. The original 19-SNP based haplotypes are also listed in Table 3, in rows corresponding to the new 12-SNP based haplotypes. The latter shows that the common haplotypes are shared between the largely Caucasian Post-CABG population and the Mexican-American cohort, albeit with modest differences in frequency.

Haplotype 12-1, the most common haplotype, was associated with protection against progression of atherosclerosis (covariate-adjusted OR 0.69, 95% CI 0.49-0.97, P=0.03); 41.4% of carriers of this haplotype experienced graft worsening compared with 48.9% of non-carriers. Furthermore, the mean proportion of grafts per subject showing progression of atherosclerosis was also significantly decreased for those carrying haplotype 12-1:27% for haplotype 12-1 carriers compared with 32% for non-carriers of this haplotype (P=0.048). Haplotype 12-1 carriers were also protected against the presence of graft occlusion (adjusted OR 0.57, 95% CI 0.36-0.91, P=0.017); 10.7% of carriers of this haplotype experienced graft occlusion compared with 16.5% of non-carriers. None of the other haplotypes were significantly associated with progression or occlusion, although haplotype 12-4 showed a trend towards more frequent graft progression (OR 1.35, 95% CI 0.84-2.17, P=0.22); 48.9% of carriers of this haplotype experienced graft worsening compared with 43.1% of non-carriers.

The fourth most frequent haplotype (12-4) was associated with a decreased increment in HDL-cholesterol (12-4 carriers: +6.8% HDL-C response vs. non-carriers: +14.3% HDL-C response, P=0.005) (Table 4). Conversely, three rare haplotypes, 12-6, 12-7, and 12-8, were each associated with increased HDLC response to therapy compared to respective non-carriers (Table 4). Haplotype 12-2 was associated with a smaller increment in triglycerides (12-2 carriers: +2.6% versus non-carriers: +11.8% change in triglycerides, P=0.02). The effects of LPL haplotypes on HDL-C and triglyceride response were independent of whether subjects were in the intensive or moderate treatment group. LPL haplotypes were not associated with TC or LDL-C response to lipid-lowering therapy.

Secondary analyses detected association of haplotype 12-1 with decreased diastolic blood pressure (DBP) at baseline (79.2±8.9 in carriers versus 80.5±8.8 mmHg in non-carriers, P=0.045), haplotype 12-4 with increased DBP (81.4±8.8 in carriers versus 79.4±8.9 mmHg in non-carriers, P=0.026), and haplotype 12-3 with increased systolic blood pressure (136.1±16.7 in carriers versus 133.1±17.5 mmHg in non-carriers, P=0.037). Haplotype 12-2 was associated with slightly decreased baseline HDL-C, 1.0±0.25 mmol/L (38.5±9.6 mg/dL) in carriers, versus 1.03±0.25 mmol/L (39.8±9.8 mg/dL) in non-carriers (P=0.035); haplotype 12-4 with increased HDL-C, 1.08±0.27 mmol/L (41.7±10.6 mg/dL) in carriers, versus 1.01±0.25 mmol/L (39.1±9.6 mg/dL) in non-carriers (P=0.013); and haplotype 12-6 with increased HDL-C, 1.09±0.29 mmol/L (42.0±11.4 mg/dL) in carriers, versus 1.01±0.25 mmol/L (39.2±9.6 mg/dL) in non carriers (P=0.032). No LPL haplotype was associated with baseline levels of TC, LDL-C, or TG.

Given the associations of haplotypes 12-1 and 12-4 with DBP, we reanalyzed the associations of these haplotypes with the primary phenotypes of atherosclerosis progression and lipid response by including DBP as a covariate in the analyses. The associations between haplotype 12-1 and atherosclerosis progression and graft occlusion were only slightly attenuated (P=0.053 and P=0.023, respectively). Inclusion of DBP as a covariate in the association analysis of haplotype 12-4 with HDL-C response did not alter the significance of that result (P=0.009).

### Example 2

TABLE 1
Clinical characteristics of the 891 post-CABG subjects

| Phenotype | Mean ± SD (range) |
|---|---|
| Age (yr) | 61.7 ± 7.5 (35.2-76.1) |
| Male gender (%) | 91.9 |
| Caucasian (%) | 93.7 |
| Current smoking (%) | 9.3 |
| Time between CABG and enrollment (yr) | 4.7 ± 2.5 (0.3-11.3) |
| Baseline systolic blood pressure (mmHg) | 134.0 ± 17.4 (82.0-200.0) |
| Baseline diastolic blood pressure (mmHg) | 79.6 ± 8.8 (55.0-108.0) |
| Baseline LDL-C (mmol/L) | 4.02 ± 0.52 (2.46-6.05) |
| Baseline HDL-C (mmol/L) | 1.02 ± 0.25 (0.49-2.56) |
| Baseline total cholesterol (mmol/L) | 5.87 ± 0.66 (3.75-8.15) |
| Baseline triglycerides (mmol/L) | 1.82 ± 0.79 (0.51-5.46) |
| LDL-C response (%) | −25.7 ± 20.6 (−78.8-45.9) |
| HDL-C response (%) | 13.8 ± 24.4 (−61.9-153.1) |
| Total cholesterol response (%) | −15.3 ± 16.3 (−58.4-43.5) |
| Triglyceride response (%) | 7.5 ± 53.0 (−81.6-330.2) |

### Example 3

TABLE 2
Frequency and position information on 12 LPL variants

| Variant | | | | |
|---|---|---|---|---|
| Designation | Variation | Location | Position | MAF |
| rs320 (8393)* | T/G | Intron 8 | −3744 | 0.28 |
| rs328 (9040)* | C/G | Exon 9 | −3097 | 0.095 |
| rs11570891 | C/T | Intron 9 | −11 | 0.10 |
| rs3289 | T/C | Exon 10/3′-UTR | 372 | 0.037 |
| rs1803924 | C/T | Exon 10/3′-UTR | 854 | 0.099 |
| rs1059507 | C/T | Exon 10/3′-UTR | 1143 | 0.18 |
| rs3735964 | C/A | Exon 10/3′-UTR | 1225 | 0.096 |
| rs3200218 | A/G | Exon 10/3′-UTR | 1251 | 0.20 |
| rs1059611 | T/C | Exon 10/3′-UTR | 1743 | 0.11 |
| rs10645926 | —/TT | Exon 10/3′-UTR | 1807 | 0.11 |
| rs15285 | C/T | Exon 10/3′-UTR | 1847 | 0.31 |
| rs3866471 | C/A | Exon 10/3′-UTR | 1849 | 0.17 |

MAF = minor allele frequency. Allele frequency data is from genotyping of 903 subjects.
Position is given to show relative distance of SNPs from one another; the numbering corresponds to the position relative to the first nucleotide of exon 10.
Numbers in parentheses correspond to the naming of SNPs in prior studies.
*rs320 is the HindIII variant; rs328 is the Ser447stop variant.

### Example 4

TABLE 3
LPL haplotypes defined by genotyping 12 polymorphisms

| Haplotypes based on 12 variants in the | | | Corresponding haplotypes | |
|---|---|---|---|---|
| Post-CABG cohort | | | from the MACAD study* | |
| Designation | Haplotype | Frequency | Designation | Frequency |
| 12-1 | 111111111111 | 0.431 | 19-1 | 0.536 |
| 12-2 | 111111121111 | 0.187 | 19-2 | 0.177 |
| 12-3 | 211112111122 | 0.168 | 19-3 | 0.107 |
| 12-4 | 222121212221 | 0.057 | 19-4 | 0.072 |
| 12-5 | 111211111111 | 0.037 | 19-6 | 0.017 |
| 12-6 | 222121112221 | 0.035 | | |
| 12-7 | 111111111121 | 0.027 | 19-5 | 0.023 |
| 12-8 | 111111211111 | 0.022 | | |

The 12-variant based haplotypes were derived from 903 genotyped subjects. 1 indicates the major allele at each SNP, 2 the minor allele.
*The Mexican-American Coronary Artery Disease (MACAD) study, a study of adult offspring of Mexican-American probands with coronary artery disease.

### Example 5

TABLE 4
Percent change in HDL-C according to LPL haplotype carrier status

| | | | P value for |
|---|---|---|---|
| Haplotype | Carriers* | Non-carriers* | association† |
| 12-1 | 12.6 ± 24.3 (568) | 15.2 ± 24.2 (261) | 0.16 |
| 12-2 | 12.4 ± 24.5 (295) | 14.0 ± 24.2 (534) | 0.44 |
| 12-3 | 14.2 ± 23.5 (262) | 13.1 ± 24.7 (567) | 0.51 |
| 12-4 | 6.8 ± 20.0 (93) | 14.3 ± 24.7 (736) | 0.0053 |
| 12-5 | 11.4 ± 25.6 (61) | 13.6 ± 24.2 (768) | 0.89 |
| 12-6 | 24.8 ± 19.8 (52) | 12.7 ± 24.4 (777) | 0.0024 |
| 12-7 | 21.9 ± 24.1 (44) | 13.0 ± 24.3 (785) | 0.012 |
| 12-8 | 23.0 ± 26.0 (43) | 12.8 ± 24.4 (786) | 0.0015 |

*Data are mean ± SD (number of subjects)
†P value for association adjusted for age, gender, current smoking status, time between CABG and enrollment, race, and lovastatin group (moderate or aggressive)

### Example 6

### Subjects

This genetic association study is ancillary to the Post-Coronary Artery Bypass Graft Trial (Post-CABG Trial) (Taylor K D, Genet Med 2004, 6: 481-486; The Post Coronary Artery Bypass Graft Trial Investigators, N Engl J Med 1997, 336: 153-162). A total of 1351 subjects from seven clinical centers throughout North America were included in the clinical trial and all were eligible as participants in this genetic ancillary study. Inclusion and exclusion criteria have been previously described (The Post Coronary Artery Bypass Graft Trial Investigators, N Engl J Med 1997, 336: 153-162). Subjects were randomly assigned for treatment to lower LDL-cholesterol levels with lovastatin, aggressively (target LDL 1.55-2.20 mmol/L (60-85 mg/dL)), cholestryramine added to lovastatin if necessary to reach target) or moderately (target LDL 3.36-3.62 mmol/L (130-140 mg/dL)). For the genetic study, DNA was isolated from 903 subjects following standard protocols. Follow-up complete angiographic data, lipid values, and DNA were available from 891 of these individuals. Subjects in this genetic study were collected in the latter years of the larger Post-CABG trial. Previous comparison of the genetic study subjects with the subjects not included in the genetic study found fewer cardiovascular events, greater aspirin use, and more favorable lipid levels in the former, suggesting that subjects with lesser risk for atherosclerosis progression were disproportionately included in the genetic study (Taylor K D, Genet Med 2004, 6: 481-486).

### Example 7

### Genotyping and Haplotype Determination

Twelve single nucleotide polymorphisms (SNPs) were genotyped for haplotype reconstruction. In this Post-CABG cohort, the HindIII polymorphism located in intron 8 (rs320, also known as 8393) was previously genotyped using conventional agarose gel techniques. Subsequently, the inventors designed PCR primers and TaqMan MGB (Applied Biosystems) probes to genotype 11 additional LPL SNPs. These were selected based on prior work demonstrating that haplotypes spanning exon 9 to exon 10 were associated with variation in post-heparin plasma LPL activity and multiple phenotypes related to the metabolic syndrome in the Mexican-American Coronary Artery Disease (MACAD) cohort (Goodarzi M O, J Clin Endocrinol Metab 2005, 90: 4816-4823). In the study of LPL in the MACAD cohort, the inventors genotyped 19 SNPs; herein, HindIII was genotyped plus a subset of 11 essential SNPs. These 11 SNPs (rs328 (Ser447Stop, also known as 9040), rs11570892, rs3289, rs1803924, rs1059507, rs3735964, rs3200218, rs1059611, rs10645926, rs15285, rs3866471) either tag the common haplotypes in this region or are unique to a particular haplotype (termed 19-4) that was associated with increased LPL activity (Goodarzi M O, J Clin Endocrinol Metab 2005, 90: 4816-4823). These 11 LPL SNPs were genotyped in 903 subjects using the 5′-exonuclease assay (TaqMan MGB).

Haploview 3 was used to determine the haplotypes present in the study population.37 Haploview constructs haplotypes by using an accelerated expectation maximization algorithm similar to the partition/ligation method (38), which creates highly accurate population frequency estimates of the phased haplotypes based on the maximum likelihood derived from the unphased input genotypes. Haploview also identified six SNPs (rs328, rs3289, rs3735964, rs3200218, rs15285, rs3866471) that tag the haplotypes with frequency>0.01.

Of the 891 subjects genotyped at all 12 LPL variants, 829 with complete follow-up phenotypic data were assigned a haplogenotype using an in-house algorithm. This algorithm examined the genotype at all six haplotype tagging SNPs for each predicted possible combination of two haplotypes (i.e. haplogenotype); the genotypes at each tag SNP (1=homozygous for major allele; B=heterozygous; 2=homozygous for minor allele) were considered together as a genotype pattern that is specific to a particular haplogenotype. In this data, each possible pair of haplotypes was reflected in a unique genotype pattern, with the exception of haplogenotype 12-1/12-4 and haplogenotype 12-6/12-8, both of which had the same genotype pattern (B1B1B1). The frequencies of these haplotypes (12-1:0.431; 12-4:0.057; 12-6:0.035; 12-8:0.022; Table 3) allowed a determination of the relative frequency of haplogenotype 12-1/12-4 versus 12-6/12-8 ((0.431×0.057)/(0.035×0.022)=32). Thus, haplogenotype 12-1/12-4 should be 32 times more common in the data than 12-6/12-8. In the inventors' population, 45 subjects had the ambiguous genotype pattern; assigning all of them a haplogenotype of 12-1/12-4 may have resulted in an error in ˜1 subject. This gives an overall error rate of 1/829 (0.12%).

### Example 8

### Phenotyping

All demographic, family history, medical history, and clinical data were collected as part of the Post-CABG Trial.14 The progression of atherosclerosis was quantitatively determined by comparison of an initial angiogram at enrollment with a follow-up angiogram repeated an average of 4.3 years later. Baseline and follow-up angiography were obtained with catheterization techniques that permitted computer-assisted quantitative measurement. An initially patent graft was defined as having progression of atherosclerosis if there was a decrease of 0.6 mm or more in lumen diameter at the site of greatest change at follow-up. Subjects with “progression of atherosclerosis” were defined as those subjects with one or more grafts showing progression. Graft occlusion was also assessed. Baseline and post-treatment lipid levels (total cholesterol, TC; LDL-cholesterol, LDL-C; HDL-cholesterol, HDL-C; and triglycerides, TG) were obtained.

All procedures were approved by the institutional review boards of Cedars-Sinai Medical Center and the other centers participating in the Post-CABG Trial. Informed consent for the clinical trial was obtained prior to enrollment and consent for this genetic study was obtained during follow-up.

### Example 9

### Association Analysis

The primary phenotypes analyzed for association with LPL haplotypes were: a) the progression of atherosclerosis, and b) lipid response to lovastatin therapy. Secondary analyses included association of the LPL haplotypes with baseline lipid and systolic and diastolic blood pressure (SBP and DBP) measurements.

Association of LPL haplotypes with presence/absence of atherosclerosis progression and with presence/absence of graft occlusion was evaluated using logistic regression. Association with TC, LDL-C, HDL-C, and TG response to lovastatin therapy, baseline lipid, and baseline blood pressure measurements was evaluated using analysis of covariance (ANCOVA). All analyses were adjusted for age, gender, current smoking status, time between CABG and enrollment, race, and lovastatin group (moderate or aggressive) by inclusion of these parameters as independent variables in the logistic regression or ANCOVA analyses. Quantitative trait values were log-transformed as appropriate to reduce nonnormality. For each haplotype analysis, haplogenotype was coded as an independent variable as “carrier” or “non-carrier.” Single SNP association analyses were not carried out, both to reduce the number of statistical tests and because our interest is in association of LPL haplotypes with atherosclerotic and metabolic phenotypes.

While the description above refers to particular embodiments of the present invention, it should be readily apparent to people of ordinary skill in the art that a number of modifications may be made without departing from the spirit thereof. The presently disclosed embodiments are, therefore, to be considered in all respects as illustrative and not restrictive. One skilled in the art will recognize many methods and materials similar or equivalent to those described herein, which could be used in the practice of the present invention. Indeed, the present invention is in no way limited to the methods and materials described. Furthermore, one of skill in the art would recognize that the invention can be applied to various metabolic conditions and disorders and diseases besides that of statin treatment and atherosclerosis. It will also be readily apparent to one of skill in the art that the invention can be used in conjunction with a variety of phenotypes, such as additional genetic variants, biochemical markers, abnormally expressed biological pathways, and various clinical manifestations.

1. A method for evaluating the prognosis of vascular grafts in an individual undergoing statin treatment, comprising:
obtaining a DNA sample from the individual; and
analyzing the DNA sample for at least one haplotype of a human gene coding lipoprotein lipase (“LPL”), the at least one haplotype selected from the group consisting of haplotype 1, haplotype 6, haplotype 7, haplotype 8, haplotype 2 and haplotype 4,
wherein the presence of haplotype 1, haplotype 6, haplotype 7, and/or haplotype 8 is indicative of a favorable prognosis, and
wherein the presence of haplotype 2 and/or haplotype 4 is indicative of an unfavorable prognosis.

2. The method of claim 1, wherein the at least one haplotype comprises SEQ. ID. NO.: 1, SEQ. ID. NO.: 2, SEQ. ID. NO.: 3, SEQ. ID. NO.: 4, SEQ. ID. NO.: 5, SEQ. ID. NO.: 6, SEQ. ID. NO.: 7, SEQ. ID. NO.: 8, SEQ. ID. NO.: 9, SEQ. ID. NO.: 10, SEQ. ID. NO.: 11, and/or SEQ. ID. NO.: 12.

3. A method of determining the prognosis of atherosclerosis in coronary grafts in an individual undergoing statin treatment, comprising:
determining the presence or absence one or more haplotype at the lipoprotein lipase (“LPL”) locus selected from the group consisting of haplotype 1, haplotype 6, haplotype 7, and haplotype 8;
determining an increase or decrease in lipid level by comparing a baseline measurement with a follow-up measurement; and
prognosing an uncomplicated case of atherosclerosis in coronary grafts if the individual undergoing statin treatment demonstrates the presence of one of the one or more haplotype at the LPL locus and/or an increase in lipid level.

4. The method of claim 3, wherein the lipid level comprises HDL-cholesterol.

5. The method of claim 3, wherein the statin is lovastatin.

6. A method of determining the prognosis of atherosclerosis in an individual undergoing statin treatment, comprising:
determining the presence or absence of one or more haplotypes at the lipoprotein lipase (“LPL”) locus selected from the group consisting of haplotype 2 and haplotype 4;
determining an increase or decrease in lipid response to statin treatment by comparing a baseline measurement with a follow-up measurement; and
prognosing a complicated case of atherosclerosis if the individual undergoing statin treatment demonstrates the presence of one of the one or more haplotypes at the LPL locus and/or a decrease in lipid response to statin treatment.

7. The method of claim 6, wherein the lipid comprises triglyceride.

8. The method of claim 6, wherein the lipid comprises HDL-cholesterol.

9. The method of claim 6, wherein the one or more haplotypes at the LPL locus comprise one or more variant alleles selected from SEQ. ID. NO.: 1, SEQ. ID. NO.: 2, SEQ. ID. NO.: 3, SEQ. ID. NO.: 4, SEQ. ID. NO.: 5, SEQ. ID. NO.: 6, SEQ. ID. NO.: 7, SEQ. ID. NO.: 8, SEQ. ID. NO.: 9, SEQ. ID. NO.: 10, SEQ. ID. NO.: 11, and SEQ. ID. NO.: 12.

10. A method of treating atherosclerosis in an individual, comprising:
determining the presence of at least one haplotype at the lipoprotein lipase locus selected from the group consisting of haplotype 2 and haplotype 4; and
treating the atherosclerosis in the individual.

11. A method of diagnosing susceptibility to vascular graft occlusion in an individual, comprising:
determining the presence or absence of haplotype 2 at the lipoprotein lipase locus and/or haplotype 4 at the lipoprotein lipase locus; and
diagnosing susceptibility to vascular graft occlusion based upon the presence of haplotype 2 at the lipoprotein lipase locus and/or haplotype 4 at the lipoprotein lipase locus.

12. A method of diagnosing a low probability of vascular graft occlusion in an individual, comprising:
determining the presence or absence of haplotype 1 at the lipoprotein lipase locus; and
diagnosing a low probability of vascular graft occlusion based upon the presence of haplotype 1.

13. The method of claim 12, wherein haplotype 1 comprises one or more variant alleles selected from SEQ. ID. NO.: 1, SEQ. ID. NO.: 2, SEQ. ID. NO.: 3, SEQ. ID. NO.: 4, SEQ. ID. NO.: 5, SEQ. ID. NO.: 6, SEQ. ID. NO.: 7, SEQ. ID. NO.: 8, SEQ. ID. NO.: 9, SEQ. ID. NO.: 10, SEQ. ID. NO.: 11, and SEQ. ID. NO.: 12.
